(19) United States
(12) Patent Application Publication
(10) Pub. No.: US 2012/0172594 A1
(43) Pub. Date: Jul. 5, 2012

(54) Process for the Synthesis of Melamine Cyanurate in Lamellar Crystalline Shape with High Purity and Flowability

(75) Inventors: Ken Zhou, Guangdong (CN); Xiuqin Zhang, Guangdon (CN); Mincong Chen, Guangdong (CN)

(73) Assignee: Presafer (Qingyan) Phosphor Chemical Company Limited, Guangdong (CN)

(21) Appl. No.: 13/212,721

(22) Filed: Aug. 18, 2011

(30) Foreign Application Priority Data
Dec. 31, 2010 (CN) 201010618165.7

(51) Int. Cl. C07D 251/70

(52) U.S. Cl. 544/198

(57) ABSTRACT

The present invention relates to a process for the synthesis of melamine cyanurate in lamellar crystalline shape with high purity and flowability. The procedures include that 1) melamine and cyanuric acid react to form mixture solution, 2) the mixture solution is filter-pressed to prepare filter cake, 3) filter cake and silicon oil are mixed to obtain semi-finished product, 4) dried the semi-finished product until the water content is less than 1.0%, 5) heat the temperature and control certain vacuum degree for curing and crystallizing, 6) the product is obtained. The processing steps of present invention are easy to operate, the processing parameters are easy to control, the production time of melamine cyanurate is short, the quantity of pure water is low, and utilization of equipments is high. Removing the incompletely reacted raw materials and impurities by filter-pressing, greatly increases the purity of semi-products.

### CROSS-REFERENCE TO RELATED APPLICATION

This application claims priority to Chinese Patent Application No. 201010618165.7, filed Dec. 31, 2010, the contents of which are incorporated herein by reference.

### TECHNICAL FIELD

The present invention relates to a process for the synthesis of melamine cyanurate

### BACKGROUND OF TECHNOLOGY

Melamine cyanurate(MCA) is a multifunctional fine chemical developed by Japan in the early 1980's. Its molecular structure is thiotrzinone molecular complex comprised by melamine and cyanuric acid through secondary bonds. The appearance of melamine cyanurate is white crystallized powder with soapy feeling. It is non-toxic, tasteless and insoluble both in water and common organic solvents. It is stable below 300° C., which begins to lyophilize and decompose to melamine and cyanuric acid at 400° C. MCA is excellent nitrogen flame retardant which is one of the perfect flame retardants for PA. Furthermore, MCA can be used as flame retardant for polyester, polyolefin, epoxy resin and polyacrylic resin. In addition, it can also be used as lubricating oil additives, coating flatting agent, electropainting plastic additives, polymer concrete additives and cosmetic raw material, etc.

With the global developing trend of halogen free flame retardant, melamine cyanurate, as an N-series flame retardant with high content of nitrogen, excellent flame retardancy, low toxicity and smoke, has created a lot of interest recently,

Now, there are three main processing routes to prepare melamine cyanurate:

### Wet Processing

This processing route to prepare melamine cyanurate is characterized in that melamine and cyanuric acid as raw materials reacted in aqueous medium. The product is obtained by adding acidic and basic reagent to adjust the pH value of the reaction mixture or adding surfactant, followed by washing, filtering, drying and grinding.

Patent DE4208027A1 discloses a process for the synthesis of melamine cyanurate. Cream suspension was formed by the reaction in the presence of water at 80° C.-100° C., under atmospheric pressure, and with high water/reactant proportion. Then the product was obtained after filtering, washing, and drying the cream suspension. The proportion water/reactant is about 9:1.

Based on the acidity, patent U.S. Pat. No. 5,202,438 develops a patent technology of melamine cyanurate compounded in strong acid water at a pH value less than 1. The advantages of this process are small amount of reaction medium (the amount of 120 parts of water can meet the total weight of 100 parts of melamine and cyanuric acid), low reaction temperature (between 80° C. and 95° C.) and short reaction time (10-30 min).

Patent CN506356A discloses the process that melamine and cyanuric acid are raw materials. The reaction is carried out in the presence of aqueous ammonia with the concentration of 2-14%, at 100-200° C., and under pressure 0.05 MPa-1.5 MPa. The melamine cyanurate, with average particle size of 5-55 μm and purity of no more than 99.9%, can be obtained.

Chinese patent CN1364858A discloses the process that equimolar mixtures of melamine and cyanuric acid, while in excess water and in the presence of some PVA, react at 90-100° C. for 1-2 h. After cooling, filtering, and drying the reactants, MCA is obtained.

### Semi-Dry Processing

Patent JP08027124 discloses a process for preparing the product that to mix the crushed melamine powder and the solid cyanuric acid powder at 120° C. Adding a little amount of water, further reacting melamine and cyanuric acid at the temperature of 350° C., finished product will be obtained after mill.

### Dry Processing

U.S. Pat. No. 5,493,023 discloses a process that melamine having an average particle size of 20 μm and cyanuric acid having an average particle size of 80 μm are mixed at room temperature. Using a jet mill, the mixture is crushed to an average particle size of about 3.94 μm. The reactant mixture is heated to 350° C. and reacts for 1 h in the electric heater. The MCA product is obtained with purity of 99.2%, recovery of 95.8% and average particle size of 4.83 μm.

Patent EP601542 discloses a process that melamine powder and cyanuric acid powder are heated at 200° C.-500° C. in the absence of any liquid medium. After heating the granulation of the mixture power, granular melamine cyanurate can be obtained.

The processing routes above for preparing melamine cyanurate have disadvantages in some degree, which results in shortcomings during production and insufficient properties of products.

Using wet processing, melamine and cyanuric acid reacted in water, the viscosity of the product is large and the solid content is only about 10%, which makes it hard to filter, Furthermore, a lot of water is consumed during processing. Using acidic and basic reagent to adjust the pH value of reactants can decrease the water/reactant proportion. However, acidic and basic material generated during processing will accelerate the corrosiveness of the equipments. Besides, using a lot of water during post processing of products will enlarge the quantity of wastewater and pollute the environment. Adding surfactant of PVA or the like can also decrease the water/reactant proportion, but thermal stability of some melamine cyanurate with surfactant decreases, because of the low thermal stability of the surfactant, which makes it hard to meet the processing condition of the engineering plastics.

The reaction rate is slow because the solubility of melamine and cyanuric acid in water are too low. Cyanuric acid includes both enol form and keto form tautomers, so that different tautomers are formed in water. The purity, morphology and crystalline structure of the product are not perfect, so the product is in the presence of a lot of irregular non-flake structure which causes sharp decrease of the mechanical properties, flame retardancy and electrical properties of flame retardant used in engineering plastics. And the application field of the plastics is greatly confined, either.

The key points of semi-dry processing are water quantity and high flowing capability of powder particles. If the water quantity is too small, the reaction rate is slow, and it's difficult to react completely, so that the purity of the products is affected. If the water quantity is too large, the fluidity of reactant powder is so poor that the reactants are agglomerated and can not react continually. Furthermore, Due to the high content of solid content of the reactants, melamine cyanurate encapsulated the surface of unreacted melamine or cyanuric acid. The purity of the products decreases, and the crystalline structure is incomplete. All the shortcomings above cause it difficult to obtain stable products by using semi-dry processing.

Dry processing has some advantages, such as simple technology and easy operation. Because the impurities in raw materials can not be discharged by other methods during dry processing, the purity of raw materials must be very high. Furthermore, the reaction temperature maintains above 350° C., so gases generated during reaction have large impact to equipments and relevant accessories. The high requirement of equipments leads to high initial investment.

### SUMMARY OF THE INVENTION

An object of the present invention is to provide a process for the synthesis of melamine cyanurate in lamellar crystalline shape with high purity and flowability.

The process for the synthesis of melamine cyanurate in lamellar crystalline shape with high purity and flowability is in the following procedures:
1) Dispersing melamine and cyanuric acid into purified water, refluxing and stirring it at the reaction temperature at 40° C.-90° C. for 1 h to 6 h to form mixture solution.
2) Filtering the mixture solution, which will creates filter cake containing 50% to 80% of solid content.
3) Feeding the filter cake into the high-speed kneader, adding silicon oil, the semi-finished product will be obtained after 1 h to 3 h of continuous stirring.
4) Putting the semi-finished product into rake dryer, increasing the temperature and the internal pressure of the rake dryer to 100° C.-150° C. and 0.1 MPa˜0.5 MPa respectively, keeping the condition for about 2 h to 4 h.
5) Controlling the water content of the semi-finished product is no more than 1.0% through setting the temperature of rake dryer at 150° C.-200° C. and the vacuum degree at 0.01 MPa˜0.1 MPa.
6) Keeping the product curing and crystallizing at 250-300° C., vacuum degree 0.01˜0.1MPa for about 2h to 4h.
7) Melamine cyanurate in lamellar crystalline shape with high purity and flowability is obtained.

Preferably, the molar ratio of melamine to cyanuric acid is 0.95-1.05:1.

Preferably, the mass ratio of sum of melamine and cyanuric acid to water is 1:2-5.

Preferably, the dosage of the silicon oil is 0.1%-1% of the dry weight of filter cake.

The silicon oil is at least one of dimethyl silicone, diethyl silicone oil, phenyl silicone oil, methylphenyl silicone oil, hydrogenous silicone oil, hydroxyl silicone oil, alkoxy silicone, acyloxy silicone oil, vinyl silicone oil, amino silicone oil and amido silicone oil.

According to the present invention, melamine cyanurate in lamellar crystalline shape with high purity and flowability can be prepared. The processing steps of present invention are easy to operate, the processing parameters are easy to control, the synthesis rate of melamine cyanurate is fast, the quantity of purified water is low, and utilization of equipments is high. Removing the incompletely reacted raw materials and impurities by filter-pressing, greatly increases the purity of semi-products. And the filtrate can be recycling used again after treatment, so that industrial wastewater is basically not generated. It effectively avoids polluting the environment, and benefits the sustainable development.

### BRIEF DESCRIPTION OF FIGURES

FIG. 1 and FIG. 2 shows scanning electron microscope photos of melamine cyanurate in lamellar crystalline shape with high purity and flowability obtained in Example 3.

FIG. 3 and FIG. 4 shows scanning electron microscope photos of melamine cyanurate compared to products obtained in Comparative Example 1.

FIG. 5 and FIG. 6 shows scanning electron microscope photos of melamine cyanurate compared to products obtained in Comparative Example 2.

### DETAILED DESCRIPTION

The process for the synthesis of melamine cyanurate in lamellar crystalline shape with high purity and flowability is characterized in the following procedures:
1) Dispersing melamine and cyanuric acid into purified water, refluxing and stirring it at the reaction temperature at 40° C.-90° C. for 1 h to 6h to form mixture solution.
2) Filtering the mixture solution, which will creates filter cake containing 50% to 80% of solid content.
3) Feeding the filter cake into the high-speed kneader, adding silicon oil, the semi-finished product will be obtained after 1 h to 3 h of continuous stirring.
4) Putting the semi-finished product into rake dryer, increasing the temperature and the internal pressure of the rake dryer to 100° C.-150° C. and 0.1 MPa˜0.5 MPa respectively, keeping the condition for about 2 h to 4 h.
5) Controlling the water content of the semi-finished product is no more than 1.0% through setting the temperature of rake dryer at 150° C.-200° C. and the vacuum degree at 0.01 MPa˜0.1 MPa.
6) Keeping the product curing and crystallizing at 250-300° C., vacuum degree at 0.01 MPa˜0.1 MPa for about 2 h to 4 h.
7) Melamine cyanurate in lamellar crystalline shape with high purity and flowability is obtained.

Preferably, the molar ratio of melamine to cyanuric acid is 0.95-1.05:1. Certainly, technicians in this field can select other proportions according to requirements.

Preferably, the mass ratio of the sum of melamine and cyanuric acid to water is 1:2-5. Certainly, technicians in this field can select other proportions according to requirements.

Preferably, the dosage of the silicon oil is 0.1%-1% of the dry weight of filter cake.

The silicon oil is at least one of dimethyl silicone, diethyl silicone oil, phenyl silicone oil, methylphenyl silicone oil, hydrogenous silicone oil, hydroxyl silicone oil, alkoxy silicone, acyloxy silicone oil, vinyl silicone oil, amino silicone oil, amido silicone oil.

Silicon oil may have the following functions:
(1) The sufficient contact of silicon oil and melamine cyanurate semi-finished product in high speed kneader decreases the viscosity degree of melamine cyanurate semi-finished product. Under certain pressure and in the presence of water at high temperature, silicon oil greatly decreases the viscosity of melamine cyanurate system, increases the flowability of the system. Furthermore, silicon oil can help those unreacted melamine and cyanuric acid in sufficient contact in water, so that the residual melamine and cyanuric acid react completely. So the purity of products is greatly increased.
(2) During the process of high temperature dehydration, because of the high temperature resistance and stability in dehydration of silicon oil, surface polarity of melamine cyanurate is decreased and the dispersion is improved. So melamine cyanurate is avoided agglomerating during the processing of high temperature dehydration.
(3) During the process of curing at high temperature, the presence of silicon oil overcomes the anisotropism of melamine cyanurate reaction, makes melamine cyanurate isotropic grow in the plane, and accelerates the transformation from acicular crystallization process to lamellar crystallization process.
(4) When the reaction finishes, silicon oil is still in melamine cyanurate. Because of the properties of silicon, melamine cyanurate prepared by the process in the present invention has high dispersity and flowability. Melamine cyanurate used in polymer materials has excellent compatibility; it can improve processing properties of polymer materials significantly, and makes the materials possess higher flame retardancy and physical properties.
(5) Because silicon oil has great dispersity to powder, the particle size of melamine cyanurate is well controlled during processing. The lamellar melamine cyanurate obtained by the process of the present invention needn't to be grinded, which avoids destroying the particle regularity of product powder by grinding, and the product can be discharged directly. The final product particles have high regularity and flowability, its particle distribution parameter is D₅₀≦3 μm and D₉₈≦25 μm.
(6) Silicon oil used in the present invention, especially amino silicone oil, has some other functions except the above excellent ones. Amino silicone oil can graft react to melamine cyanurate, so the product obtained is melamine cyanurate flame retardant which has siloxane function.

In the technology schemes of the present invention, reacting at high temperature and high pressure in rake dryer has the following functions:
(1) The semi-finished product is fed in rake dryer. Compared with other conditions, unreacted melamine and cyanuric acid can react completely at high temperature and high pressure.
(2) The solid content of semi-finished product is high, so reacting at high temperature and high pressure in rake dryer greatly decreases the energy consumption. And the high concentration of semi-finished product greatly increased the reaction speed of melamine cyanurate.

The present invention will hereinafter be described more specifically by the following examples.

### EXAMPLE 1

1) In a reaction kettle with stirring apparatus and condensing unit having an internal capacity of 500 liters, 30.87 kg of melamine and 31.61 kg of cyanuric acid (the molar ratio of melamine and cyanuric acid is 1:1) are substantially dispersed in 250 liters of purified water, followed by mixing at 60° C. for 3 h to form mixture solution.
2) Filter-pressing the mixture solution for getting the filter cake by solid content of 60%.
3) Feeding the filter cake in high speed kneader. During the stirring process, dimethyl silicone is added in an amount of 0.3% based on the dry weight of filter cake. The semi-finished product is obtained after stirring for 1 h.
4) Putting the semi-finished product into rake dryer. Heating the temperature of rake dryer to 120° C., and controlling internal pressure at 0.2 MPa for 3 h.
5) Setting the temperature of the rake dryer at 190° C. and vacuum degree at 0.08 MPa for 6 h, the water content of the product dried in this way is 0.9%.
6) Keeping the product curing and crystallizing at 260° C., vacuum degree at 0.08 MPa for 3 h.
7) 62 kgs melamine cyanurate in lamellar crystalline shape with high purity and flowability are obtained.

After tested, we found that the purity of the final product is 99.85%, residual melamine is 0.04%, residual cyanuric acid is 0.04%, water content is 0.07%, the particle size is D₅₀=2.3 μm, D₉₈20.1 μm, initial decomposition temperature is 301.5° C., 1% decomposition temperature and 5% decomposition temperature are 308.1° C. and 339.3° C. respectively.

### EXAMPLE 2

1) In a reaction kettle with stirring apparatus and condensing unit having an internal capacity of 1000 liters, 74.12 kg of melamine and 75.88 kg of cyanuric acid (the molar ratio of melamine and cyanuric acid is 1:1) are substantially dispersed in 500 liters of pure water, followed by mixing at 80° C. for 3.5 h to form mixture solution.
2) Filter-pressing the mixture solution for getting the filter cake by solid content of 65%.
3) Feeding the filter cake in high speed kneader. During the stirring process, dimethyl silicone is added in an amount of 0.8% based on the dry weight of filter cake. The semi-manufactured product is obtained after stirring for 1.5 h.
4) Putting the semi-finished product into rake dryer. Heating the temperature of rake dryer to 150° C., and controlling internal pressure at 0.4 MPa for 2 h.
5) Setting the temperature of the rake dryer at 160° C. and vacuum degree at 0.06 MPa for 5 h, the water content of the product dried in this way is 0.7%.
6) Keeping the product curing and crystallizing at 270° C., vacuum degree at 0.06 MPa for 2 h.
7) 142.50 kgs melamine cyanurate in lamellar crystalline shape with high purity and flowability are obtained.

After tested, we found that the purity of the final product is 99.9%, residual melamine is 0.02%, residual cyanuric acid is 0.02%, water content is 0.06%, the particle size is D₅₀=2.5 μm, D₉₈=21.3 μm, initial decomposition temperature is 301.3° C., 1% decomposition temperature and 5% decomposition temperature are 308.1° C. and 339.2° C. respectively.

### EXAMPLE 3

1) In a reaction kettle with stirring apparatus and condensing unit having an internal capacity of 3000 liters, 290.50 kg of melamine and 303.50 kg of cyanuric acid (the molar ratio of melamine and cyanuric acid is 0.98:1) are substantially dispersed in 1800 liters of pure water, followed by mixing at 50° C. for 2 h to form mixture solution.
2) Filter-pressing the mixture solution for getting the filter cake by solid content of 70%.
3) Feeding the filter cake in high speed kneader. During the stirring process, dimethyl silicone is added in an amount of 0.6% based on the dry weight of filter cake. The semi-manufactured product is obtained after stirring for 2 h.
4) Putting the semi-finished product into rake dryer. Heating the temperature of rake dryer to 130° C., and controlling internal pressure at 0.3 MPa for 3 h.
5) Setting the temperature of the rake dryer at 180° C. and vacuum degree at 0.04 MPa for 7 h, the water content of the product dried in this way is 0.1%.
6) Keeping the product curing and crystallizing at 280° C., vacuum degree at 0.04 MPa for 4 h.
7) 576.18 kgs melamine cyanurate in lamellar crystalline shape with high purity and flowability are obtained.

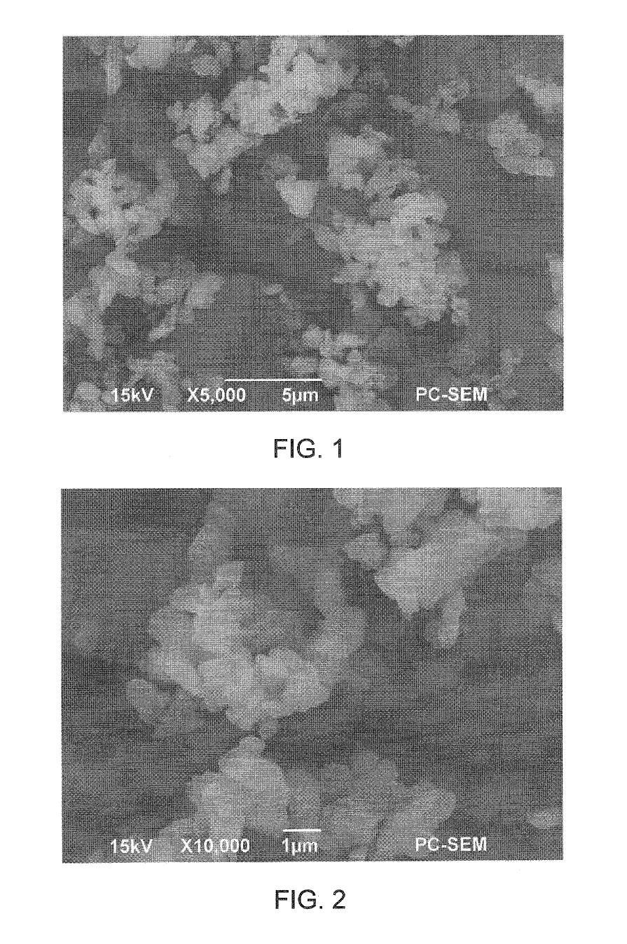

After tested, we found that the purity of the final product is 99.9%, residual melamine is 0.03%, residual cyanuric acid is 0.02%, water content is 0.05%, the particle size is D₅₀=1.9 μm, D₉₈=20.0 μm, initial decomposition temperature is 301.3° C., 1% decomposition temperature and 5% decomposition temperature are 308.2° C. and 341.9° C. respectively. The scanning electron microscope photos of melamine cyanurate product will be shown in FIG. 1 and FIG. 2.

### EXAMPLE 4

1) In a reaction kettle with stirring apparatus and condensing unit having an internal capacity of 5000 liters, 400.00 kg of melamine and 404.70 kg of cyanuric acid (the molar ratio of melamine and cyanuric acid is 1.01:1) are substantially dispersed in 2800 liters of pure water, followed by mixing at 70° C. for 4 h to form mixture solution.
2) Filter-pressing the mixture solution for getting the filter cake by solid content of 65%.
3) Feeding the filter cake in high speed kneader. During the stirring process, dimethyl silicone is added in an amount of 0.9% based on the dry weight of filter cake. The semi-manufactured product is obtained after stirring for 2.5 h.
4) Putting the semi-finished product into rake dryer. Heating the temperature of rake dryer to 110° C., and controlling internal pressure at 0.15 MPa for 4 h.
5) Setting the temperature of the rake dryer at 175° C. and vacuum degree at 0.05 MPa for 6 h, the water content of the product dried in this way is 0.6%.
6) Keeping the product curing and crystallizing at 290° C., vacuum degree at 0.05 MPa for 2 h.
7) 772.51 kgs melamine cyanurate in lamellar crystalline type with high purity and flowability are obtained.

After tested, we found that the purity of the final product is 99.81%, residual melamine is 0.06%, residual cyanuric acid is 0.05%, water content is 0.08%, the particle size is D₅₀=2.5 μm, D₉₈=22.3 μm, initial decomposition temperature is 300.5° C., 1% decomposition temperature and 5% decomposition temperature are 308.4° C. and 340.2° C. respectively.

### COMPARATIVE EXAMPLE 1

1) In a reaction kettle with stirring apparatus and condensing unit having an internal capacity of 500 liters, 30.87 kg of melamine and 31.61 kg of cyanuric acid (the molar ratio of melamine and cyanuric acid is 1:1) are substantially dispersed in 250 liters of pure water, followed by mixing at 60° C. for 3 h to form mixture solution.
2) The mixture solution is filter-pressed to prepare filter cake. The filter cake is dried in oven at 120° C., then 59.36 kg melamine cyanurate is obtained.

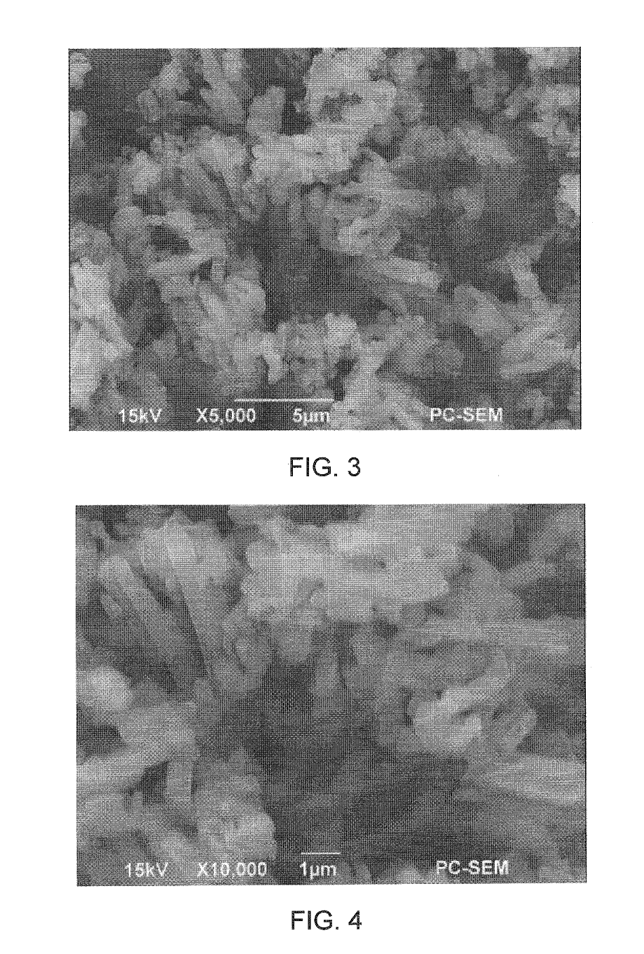

After tested, we found that the purity of the final product is 98.1%, residual melamine is 1%, residual cyanuric acid is 0.78%, water content is 0.12%, the particle size is D₅₀=3.9 μm, D₉₈=28.3 μm, initial decomposition temperature is 300.1° C., 1% decomposition temperature and 5% decomposition temperature are 307.1° C. and 339.7° C. respectively. The granule morphology is acicular, and the scanning electron microscope photos of melamine cyanurate will be shown in FIG. 3 and FIG. 4

### COMPARATIVE EXAMPLE 2

1) In a reaction kettle with stirring apparatus and condensing unit having an internal capacity of 500 liters, 30.87 kg of melamine and 31.61 kg of cyanuric acid (the molar ratio of melamine and cyanuric acid is 1:1) are substantially dispersed in 250 liters of pure water, followed by mixing at 60° C. for 3 h to form mixture solution.
2) The mixture solution is filter-pressed to prepare semi-manufactured product by solid content of 60%.
3) Putting the semi-finished product into rake dryer. Jacket is used for the rake dryer to heat the heat-conducting oil. Maintaining the temperature of heat-conducting oil in rake dryer at 190° C. and internal pressure at 0.08MPa for 5 h.
4) Heating the temperature of heat-conducting oil to 260° C. and controlling the vacuum degree of the rake dryer at 0.08 MPa, wherein the product is curing and crystallizing for 3 h.
5) 62 kgs melamine cyanurate in lamellar crystalline type with high purity and flowability are obtained.

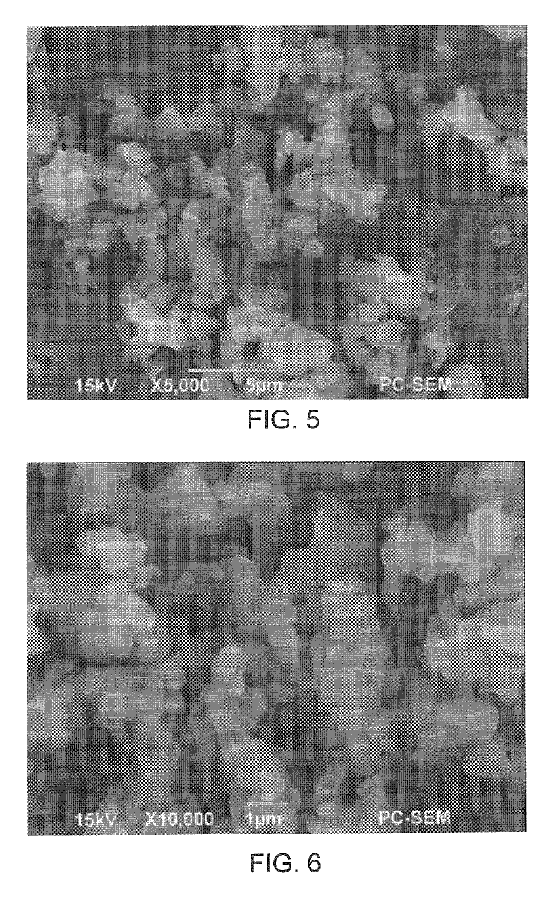

After tested, we found that the purity of the final product is 99.1%, residual melamine is 0.45%, residual cyanuric acid is 0.35%, water content is 0.1%, the particle size is D₅₀=3.5 μm, D₉₈=26.8 μm, initial decomposition temperature is 301.2° C., 1% decomposition temperature and 5% decomposition temperature are 308.1° C. and 340.9° C. respectively. The granule morphology is acicular, and the scanning electron microscope photos of melamine cyanurate will be shown in FIG. 5 and FIG. 6

Only limited types of silicon oil are used in the examples above, but if technicians in this field know the generality of silicon oils, they can select other silicon oils in the technical scheme of the present patent without doing any creative work. Certainly, all these replacements are in the scope of the present patent.

I claim:

1. The process for the synthesis of melamine cyanurate in lamellar crystalline shape with high purity and flowability is characterized in the following procedures:
1) Dispersing melamine and cyanuric acid into purified water, refluxing and stirring it at the reaction temperature at 40° C.-90° C. for 1 h to 6h to form mixture solution.
2) Filtering the mixture solution, which will creates filter cake containing 50% to 80% of solid content.
3) Feeding the filter cake into the high-speed kneader, adding silicon oil, the semi-finished product will be obtained after 1 h to 3 h of continuous stirring.
4) Putting the semi-finished product into rake dryer, increasing the temperature and the internal pressure of the rake dryer to 100° C.-150° C. and 0.1 MPa-0.5 MPa respectively, keeping the condition for about 2 h to 4 h.
5) Controlling the water content of the semi-finished product is no more than 1.0% through setting the temperature of rake dryer at 150° C.-200° C. and the vacuum degree at 0.01 MPa˜0.1 MPa.
6) Keeping the product curing and crystallizing at 250-300° C., vacuum degree 0.01˜0.1 MPa for about 2 h to 4 h.
7) Melamine cyanurate in lamellar crystalline shape with high purity and flowability is obtained.

2. A process for the synthesis of melamine cyanurate in lamellar crystalline shape with high purity and flowability according to claim 1, the characteristic is: the molar ratio of melamine to cyanuric acid is 0.95-1.05:1.

3. A process for the synthesis of melamine cyanurate in lamellar crystalline shape with high purity and flowability according to claim 1, the characteristic is: the mass ratio of sum of melamine and cyanuric acid to water is 1:2-5.

4. A process for the synthesis of melamine cyanurate in lamellar crystalline shape with high purity and flowability according to claim 1, wherein silicon oil is added in an amount of 0.1-1% based on the dry weight of filter cake.

5. A process for the synthesis of melamine cyanurate in lamellar crystalline shape with high purity and flowability according to claim 1, wherein the silicon oil is at least one of dimethyl silicone, diethyl silicone oil, phenyl silicone oil, methylphenyl silicone oil, hydrogenous silicone oil, hydroxyl silicone oil, alkoxy silicone, acyloxy silicone oil, vinyl silicone oil, amino silicone oil and amido silicone oil.
